(19) United States
(12) Patent Application Publication
(10) Pub. No.: US 2026/0076720 A1
(43) Pub. Date: Mar. 19, 2026

(54) SENSING MULTI AXIAL SCREW HEAD

(71) Applicant: Warsaw Orthopedic, Inc., Warsaw, IN (US)

(72) Inventors: Arjun Siby Kurian, Chesterfield, MO (US); Adam D. Glaser, Collierville, TN (US); Newton H. Metcalf, JR., Memphis, TN (US); Keith E. Miller, Germantown, TN (US)

(21) Appl. No.: 19/321,422

(22) Filed: Sep. 8, 2025

Related U.S. Application Data
(60) Provisional application No. 63/694,580, filed on Sep. 13, 2024.

(51) Int. Cl. A61B 17/70; A61B 17/00; A61B 90/00

(52) U.S. Cl. CPC A61B 17/7001; A61B 90/06; A61B 2017/00084; A61B 2017/00221; A61B 2017/00477; A61B 2017/00734; A61B 2017/00951; A61B 2090/064

(57) ABSTRACT

Load-sensing implants, sensor packages, and surgical-site monitoring systems are disclosed. A load-sensing implant includes an attachment portion configured to secure the implant to a bone of a subject. The implant further includes a receiver portion configured to receive and secure a longitudinal member and an electronics enclosure disposed adjacent the receiver portion and forming a common wall with the receiver portion. The electronics enclosure includes a sensor package comprising multiple strain sensors disposed on a backing, the sensor package configured to sense, via the common wall, forces applied to the receiver portion by the longitudinal member and support electronics. The support electronics are configured to receive, from the sensor package, data representing the sensed forces, and transmit the received data to an external device.

### CROSS-REFERENCE TO RELATED APPLICATION

This application claims the benefit of and priority to U.S. Provisional Ser. No. 63/694,580 filed Sep. 13, 2024, the entire disclosure of which is incorporated by reference herein.

### BACKGROUND

The present disclosure generally relates to mechanical and electrical sensor assemblies for implant devices, and more particularly to implant systems including a multi-axis sensor package.

Treatments of spinal disorders, such as degenerative disc disease, disc herniations, scoliosis or other curvature abnormalities, and fractures, often require surgical treatments. For example, spinal fusion may be used to limit motion between vertebral members. As another example, implants may be used to preserve motion between vertebral members.

Surgical treatment typically involves the use of longitudinal members, such as spinal rods. Longitudinal members may be attached to the exterior of two or more vertebral members to assist with the treatment of a spinal disorder. Longitudinal members may provide a stable, rigid column that helps bones to fuse, and may redirect forces over a wider area away from a damaged or defective region. Also, rigid longitudinal members may help in spinal alignment.

Screw assemblies may be used to connect a longitudinal member to a vertebral member. A screw assembly may include a pedicle screw, hook, or other connector, among other components. A pedicle screw can be placed in, above and/or below vertebral members that were fused, and a longitudinal member can be used to connect the pedicle screws which inhibits or controls movement. A set screw can be used to secure the connection of a longitudinal member to a pedicle screw, hook or other connector. However, the connection force and continued integrity of the connection between a longitudinal member and a pedicle screw or other connector can be challenging to monitor during and after implantation. In addition, it is difficult to monitor that an appropriate force is maintained between an implant and a longitudinal member. In particular, there is a need for sensor packages that allow implants to effectively sense forces applied by a longitudinal member along multiple axes.

This document describes methods and systems that are directed to addressing the problems described above, and/or other issues.

### SUMMARY

This disclosure generally relates to load-sensing implants, sensor packages, and surgical-site monitoring systems. Issues associated with prior solutions are addressed by the subject matter of the independent claims included in this document. Additional advantageous aspects are included in the dependent claims.

In one aspect, the present disclosure provides a load-sensing implant. The load-sensing implant includes an attachment portion configured to secure the implant to a bone of a subject. The implant further includes a receiver portion configured to receive and secure a longitudinal member and an electronics enclosure disposed adjacent the receiver portion and forming a common wall with the receiver portion. The electronics enclosure includes a sensor package comprising multiple strain sensors disposed on a backing, the sensor package configured to sense, via the common wall, forces applied to the receiver portion by the longitudinal member and support electronics. The support electronics are configured to receive, from the sensor package, data representing the sensed forces, and transmit the received data to an external device.

Implementations of the disclosure may include one or more of the following optional features. In some examples, the sensor package is disposed in a recessed pocket formed in the common wall. The recessed pocket may be formed to enhance transfer of forces from the receiver portion to the sensor package. The sensor package may include multiple strain gauges. In some examples, the sensor package includes two strain gauges oriented to sense shear forces and two strain gauges positioned to sense axial forces. Each sensor of the sensor package may have an impedance of at least 1000 ohms. In some examples, the electronics enclosure is capped with a header configured to enhance wireless communication between the implant and the external device. The electronics enclosure may further contain a power source. The sensor package may be attached to the common wall by an adhesive applied to the backing.

In another aspect, the present disclosure provides a sensor package for a spinal implant. The sensor package includes a flexible backing, two outer strain gauges attached to the backing and having a sensing element with a longitudinal axis, the longitudinal axis oriented along a first direction, a first inner strain gauge attached to the backing and having a sensing element with a longitudinal axis with the longitudinal axis oriented at 45 degrees from the first direction, and a second inner strain gauge attached to the backing and having a sensing element with a longitudinal axis oriented at 45 degrees from the first direction and 90 degrees from the longitudinal axis of the sensing element of the first inner strain gauge. Each sensing element has an impedance of at least 1000 ohms.

Implementations of the disclosure may include one or more of the following optional features. In some examples, the sensor package further includes an adhesive applied to the backing. In some examples, each sensing element has an impedance of at least 1000 ohms.

In another aspect, the present disclosure provides a surgical site (SS) monitoring system. The SS monitoring system includes an external reader device and one or more load-sensing implants. Each implant includes an attachment portion configured to secure the implant to a bone of a subject, a receiver portion configured to receive and secure a longitudinal member, and an electronics enclosure disposed adjacent the receiver portion and forming a common wall with the receiver portion. The electronics enclosure contains a sensor package comprising multiple strain sensors disposed on a backing, the sensor package configured to sense, via the common wall, forces applied to the receiver portion by the longitudinal member. The electronics enclosure further contains support electronics configured to receive, from the sensor package, data representing the sensed forces, and transmit the received data to the external reader device.

Implementations of the disclosure may include one or more of the following optional features. In some examples, the sensor package is disposed in a recessed pocket formed in the common wall. The recessed pocket may be formed to enhance transfer of forces from the receiver portion to the sensor package. The sensor package may include multiple strain gauges. In some examples, the sensor package includes two strain gauges oriented to sense shear forces and two strain gauges positioned to sense axial forces. Each sensor of the sensor package may have an impedance of at least 1000 ohms. In some examples, the electronics enclosure is capped with a header configured to enhance wireless communication between the implant and the external reader device. At least one of the one or more load-sensing implants may further include a temperature gauge or a position sensor.

### BRIEF DESCRIPTION OF THE DRAWINGS

The accompanying drawings are incorporated into this document and form a part of the specification.

FIG. 1 illustrates an example load-sensing implant.

FIG. 2 shows an example environment for installing load-sensing implants.

FIG. 3 illustrates an example sensor package.

FIG. 4 shows an exploded view of the example implant.

FIG. 5 shows an example sensor package epoxied in place.

FIG. 6 shows another view of an example implant.

FIG. 7 illustrates an example of a surgical site monitoring system according to an embodiment.

In the drawings, like reference numbers generally indicate identical or similar elements. Additionally, generally, the left-most digit(s) of a reference number identifies the drawing in which the reference number first appears.

### DETAILED DESCRIPTION

The exemplary embodiments of the surgical system and related methods of use disclosed are discussed in terms of medical devices for the treatment of musculoskeletal disorders and more particularly, in terms of vertebral fixation screws, including for example pedicle screws, as well as hooks, cross connectors, offset connectors and related systems for use during various spinal procedures or other orthopedic procedures and that may be used in conjunction with other devices and instruments related to spinal treatment, such as rods, wires, plates, intervertebral implants, and other spinal or orthopedic implants, insertion instruments, specialized instruments such as, for example, delivery devices (including various types of cannula) for the delivery of these various spinal or other implants to the vertebra or other areas within a patient in various directions, and/or a method or methods for treating a spine, such as open procedures, mini-open procedures, or minimally invasive procedures. Exemplary prior art devices that may be modified to include the various embodiments of load sensing systems include, for example, U.S. Pat. Nos. 6,485,491 and 8,057,519, incorporated herein by reference in their entirety.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting.

In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value.

Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other and are not necessarily “superior” and “inferior.” Generally, similar spatial references of different aspects or components indicate similar spatial orientation and/or positioning, i.e., that each “first end” is situated on or directed towards the same end of the device.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

The following discussion includes a description of an implant system having sensing capability as well as related components and methods of employing the implant in accordance with the principles of the present disclosure. Reference is made in detail to the exemplary embodiments of the present disclosure, which are illustrated in the accompanying figures.

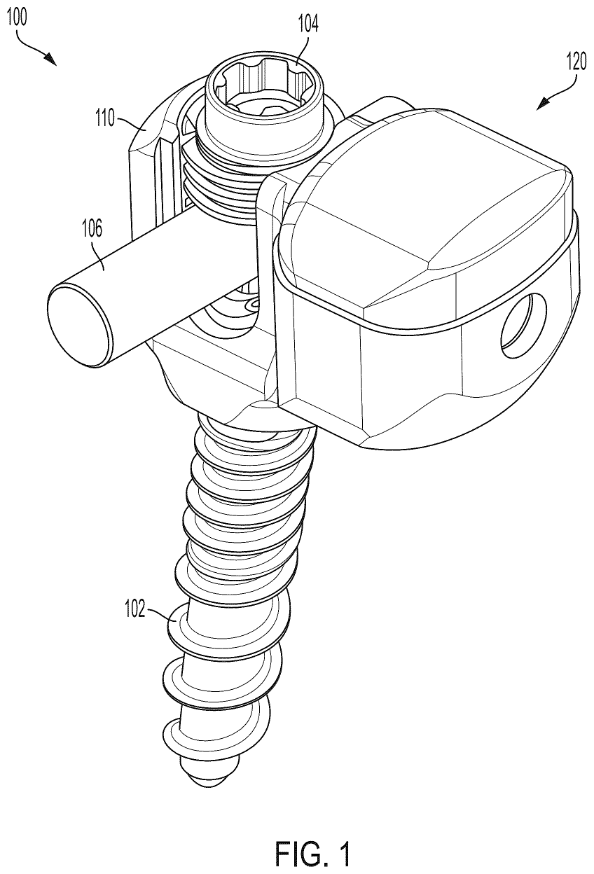

Referring to FIG. 1, an example load-sensing implant 100 is shown. The implant 100 includes an attachment portion 102 for securing/anchoring the implant 100 to a bone of a subject. For example, the attachment portion 102 may include a screw configured to anchor the implant 100 to a pedicle of a vertebra. In some examples, the attachment portion 102 is rigidly connected to the implant 100. That is, the implant may have a fixed-angle screw. In other examples, the attachment portion 102 has a range of motion along several different axes relative to the implant 100. An example of the latter embodiment is a multiaxial pedicle screw. The attachment portion 102 is not limited to pedicle screws. In other examples, the attachment portion 102 may include a hook member, a cross-link connector, an offset connector, or a hybrid hook-screw member, or other mechanism for securely fastening the implant 100 to the patient's anatomy (in either a polyaxial or multiaxial configuration).

The implant 100 also includes a receiver portion 110 configured to receive and secure a longitudinal member 106 such as a spinal rod. As shown, the longitudinal member 106 is secured within the receiver portion 110 of the implant 100 by a set screw 104. By driving the set screw 104, the longitudinal rod 106 may be clamped between the set screw 104 and the body of the implant 100. In other examples, the receiver portion 110 configured to secure a transverse connector, such as a cross-link between spinal rods in a construct. The longitudinal member 106 may be used to connect adjacent implants 100, each of which may be secured to a patient's bone (each, adjacent vertebrae) and also secured within a respective implant 100. Similarly, a cross-link may be used to connect adjacent spinal rods, each of which may be secured to a patient's bone via additional implants 100.

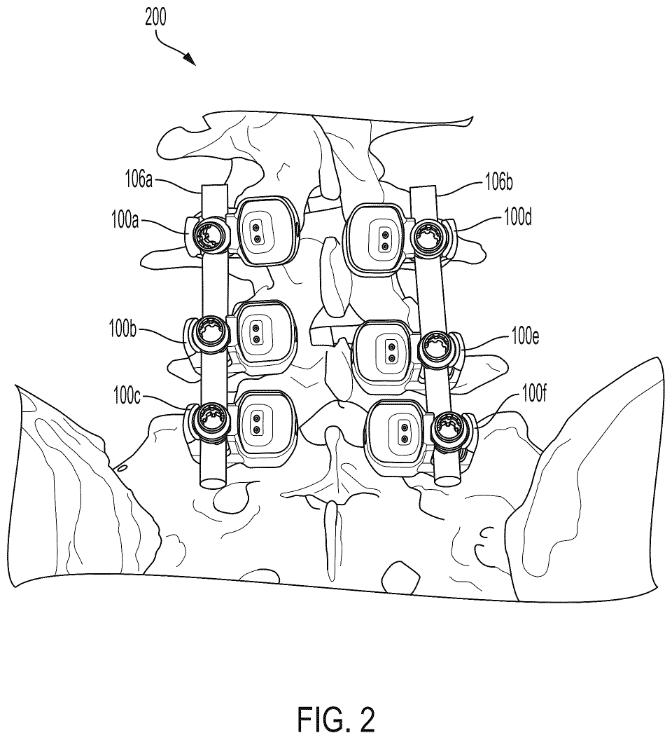

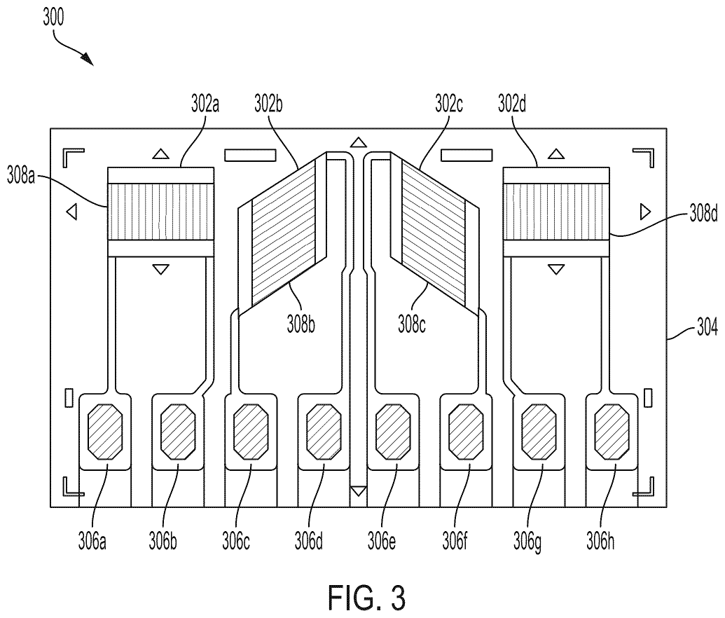

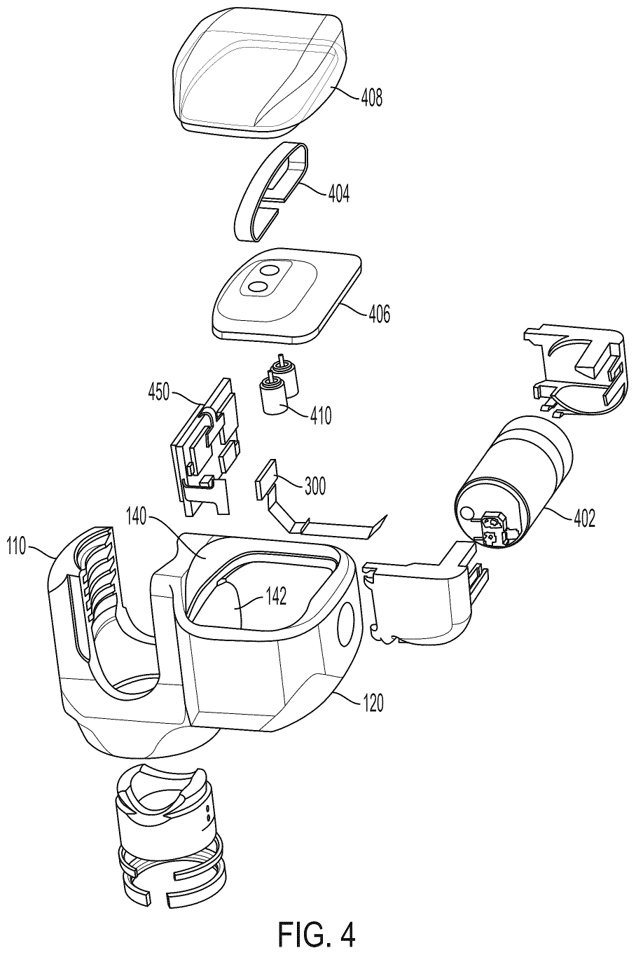

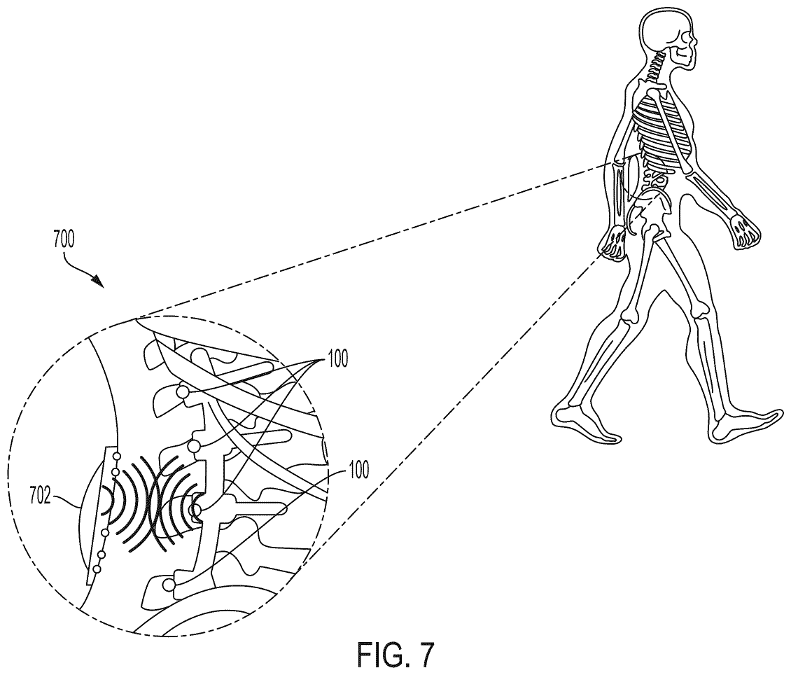

FIG. 2 shows an example environment 200 for installing load-sensing implants 100. A surgeon or other medical professional may install a construct including multiple implants 100a-100f and one or more longitudinal members 106a, 106b to add extra support and strength to the patient's spine, and/or prevent movement of vertebrae, e.g., to allow a spinal fusion to heal. The surgeon may also use the construct to maintain or correct anatomic alignment of spinal segments by distributing the loads acting on the spine. In these and other cases, the surgeon may want to monitor the forced applies by the longitudinal member(s) 106 to the implants 100. For example, an abrupt change in forces applied to an implant 100 may indicate a bone fracture or a malfunction of the construct, such as loosening or dislodgement of one or more implants 100. Even in the absence of a malfunction, the surgeon may want to monitor, e.g., fusion status against the surgeon's expectations. To this end, the load-sensing implant 100 includes an electronics enclosure 120 (FIG. 1) that houses sensors 302 (FIG. 3), readout electronics 450 (FIG. 4), and other associated support electronics, e.g., 402, 410, 404 (FIG. 4). The associated support electronics are configured to transmit sensor data to an external device, e.g., 702 (FIG. 7). In some examples, some or all of the sensors 302 are integrated in a sensor package 300 (FIG. 3) or sensor assembly. The sensor package 300 is configured to sense, via the common wall 140, forces applied to the receiver portion 110 by the longitudinal member 106.

Referring to FIG. 3, an example sensor package 300 is shown. The example sensor package 300 includes four 4 sensors 302a-d on a backing 304. Each sensor 302 includes a sensing element 308a-d as well as leads 306a-306h for interfacing with the associated readout electronics 450. As shown, each sensing element 308 is a strain gauge, i.e., a device whose resistance varies with deformation. This resistance change is typically measured using a Wheatstone bridge. Strain gauges are typically configured to be more sensitive to strain in particular directions, such as the longitudinal axis of a grid pattern. As shown in FIG. 3, the grid patterns of the outer strain gauges 302 have longitudinal axes in the “vertical” direction (i.e., along an axis parallel with the central axis of the set screw 104), whereas the longitudinal axes of the inner strain gauges 302 have longitudinal axes at 45 degrees from the vertical axis. In this arrangement, the sensor package 300 as a whole may be able to sense both shear and axial forces applied to the implant 100. That is, the example sensor arrangement of FIG. 3 is configured to simultaneously measure forces aligned with the axis of the set screw 104 as well as forces aligned with the axis of the longitudinal rod 106. Other sensor 302 arrangements are also possible and may be optimized for the expected forces in particular applications. In some examples, the aggregate sensing capability is optimized for the ability to measure forces on the implant during activities of daily living (such as walking) and/or during flexion/extension, lateral bending and axial rotation (e.g., through a typical range of motion).

For example, the inner strain gauges 302 have longitudinal axes at greater than 45 degrees from the vertical axis or less than 45 degrees in order to have greater sensitivity to shear forces or greater sensitivity to axial forces. In other embodiments, the outer strain gauges 302 may be angled in lieu of (or in addition to) the inner strain gauges. Furthermore, the total number of sensors 302 may be greater than or less than four without departing from the scope of the disclosure.

As shown, all four strain gauges 302 share a single, backing 304, which makes the process of installation easier. That is, all the sensors 302 can be installed simultaneously by attaching the backing 304, as opposed to attaching sensors 302 individually. Furthermore, the sensing elements 308 can be more precisely aligned during fabrication of the sensor package 300, so that when the backing 304 is attached to the shared wall 140, each of the sensors 302 is properly aligned relative to the expected forces. The backing 304 may be attached to the common wall 140 by a suitable adhesive, such as cyanoacrylate, and may be flexible to effectively transfer strain from the common wall 140 to the sensors 302.

The associated readout electronics 450 may interface with each sensor 302 individually, e.g., in a quarter Wheatstone bridge configuration. In other embodiments, the readout electronics 450 may interface with pairs of sensors 302, e.g., in series or in parallel, or in a half Wheatstone bridge configuration. This latter configuration may provide for increased sensitivity. For example, in the sensor package 300 of FIG. 3, one half-bridge may include the outer sensors 302a, 302d, and a second half-bridge may include the inner sensors 302b, 302c. This configuration may be more sensitive to forces that act differentially on the sensors 302. That is, this configuration may be more sensitive to forces that compress sensing element 308a (deceasing its resistance) and stretch sensing element 308d (increasing its resistance) and vice versa. However, this configuration may be less sensitive to forces that equally compress or stretch both outer and inner sensors 302a-302d. In some examples, all four sensors 302 are configured in a full Wheatstone bridge.

As shown in FIG. 4, the readout electronics 450 may powered by a battery 402 or other source of electrical power. The power consumed by the sensors 302 will be a function of their individual resistance and their readout configuration (e.g., quarter Wheatstone, half Wheatstone, etc.). Therefore, the lifetime of the battery 402 will also be a function of those factors. To ensure a sufficiently long battery 402 lifetime to last the duration of a post-operative recovery period, the individual resistance of the sensing elements 308 may be configured to be at least a threshold value but which also provides sufficient sensitivity to measure clinically relevant forces/loads and/or detect clinical events such as fusion progression and/or hardware failure. In one example, a per-element resistance of about 1000 ohms was found to provide a functional device lifespan of two years.

Referring to FIG. 4, an exploded view of the example implant 100 is shown. In this view, the common wall 140 shared between the receiver portion 110 and electronics enclosure 120 can be seen. In this embodiment, the receiver portion 110 and electronics enclosure 120 (and the common wall 140) are fully integrated. That is, they are formed from the same material into a single part. In other examples, the electronics enclosure 120 and receiver portion 110 may be separately formed and subsequently attached together, e.g., by welding or epoxy, such that they share the common wall 140. In these and other examples, forces applied by the longitudinal rod 106 to the implant 100 are transferred to the common wall 140 where they can be detected by the sensor package 300. As shown, a recessed pocket 142 is formed in the common wall 140. That is, the common wall 140 has been modified to include a recessed feature having a geometric shape. As shown, the recessed pocket 142 is roughly rectangular, but other shapes are also within the scope of the disclosure. Furthermore, parameters such as length, width, depth, corner radii, and the like, may all be adjusted to fine-tune the transfer of forces to the sensor package 300. Furthermore, any of these characteristics may vary at different positions of the pocket 142. For example, the depth of the pocket 142 may vary across the width of the pocket 142, e.g., to be greater near the middle compared to the edges (or vice versa) in order to fine-tune the response of the sensors 302.

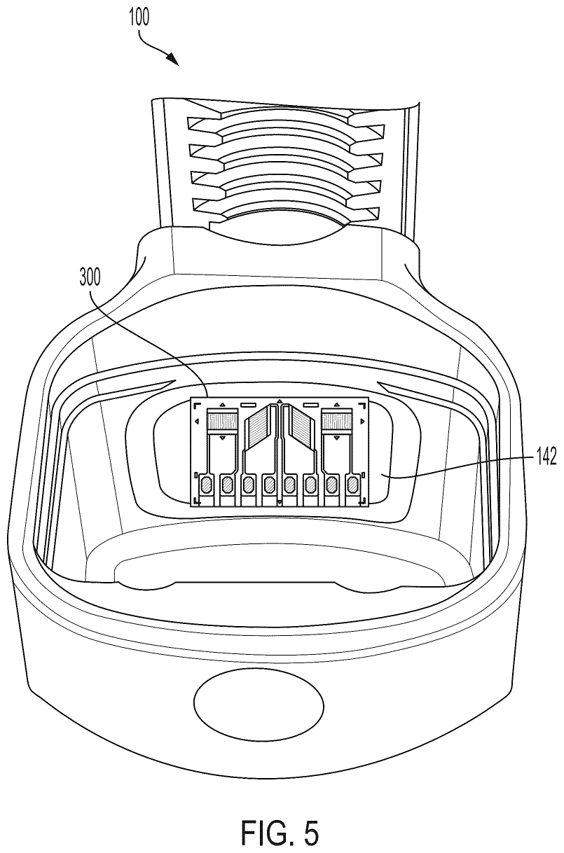

The recessed pocket 142 may provide additional room to accommodate the sensor package 300. The recessed pocket 142 may also be shaped to focus or amplify the strain on the portion of the shared wall 140 where the sensors 302 are attached. As shown, the recessed pocket 142 has rounded corners and a substantially flat portion where the sensor package 300 can be attached. Furthermore, by thinning the common wall 140, the recessed pocket 142 may permit forces to be more directly transferred to the sensors 302. That is, the thinner wall 140 may deform more readily when forces are applied, allowing the sensors 302 to more easily sense the deformation. Referring to FIG. 5, an example sensor package 300 is shown epoxied in place within the recessed pocket 142 of the common wall 140.

Referring back to FIG. 4, the support electronics associated with sensor package 300 may include a battery 402 or other power source, and readout electronics 450. These components may be hermetically sealed within the electronics housing, e.g., by cover 406. As shown, an antenna 404 is located on the patient-facing side of cover 406, to allow for more effective transmission of sensor data to an external reader 702. The antenna 404 is enclosed within a header or cap 408 that is configured to protect the antenna 404 and to protect tissue of the patient from damage/irritation from the antenna 404. The readout electronics 450 interface with the antenna 404 via feedthroughs 410 that pass through the cover 406. The example support electronics may include various electronic components in electrical communication with one another. For example, the readout electronics 450 may include a mainboard or other suitable printed circuit board (PCB), which may be electrically connected to an application specific integrated circuit (ASIC), a transceiver, a charge storage capacitor, and various mechanical electrical sensors (MEMs) such as temperature sensors, position sensors, gyroscopes, and the like. In some embodiments, electronics components may include a pre-packaged self-contained unit that is attached to the electronics enclosure 120 by, e.g., adhesive, chemical, mechanical or cement bonding. Additionally, electronics components may include a non-transitory data store (not illustrated) according to an embodiment, e.g., a memory cell such as a solid-state memory cell or the like. The non-transitory memory data store may store information and/or data from various MEMs sensors 302, for example. A non-transitory data store may be used to store various information. For example, one or more measurements of a strain gauge 302 may be stored in memory. As another example, a unique identifier associated with a load sensing assembly, a component thereof, or the implant 100 may be stored in memory. Additional and/or alternate information or types of information may be stored as is consistent with this disclosure. Additionally, in some embodiments, electronics components may be coated in a material to prevent and/or suppress corrosion, e.g., a conformal coating, an epoxy coating, aerosol coating, or the like.

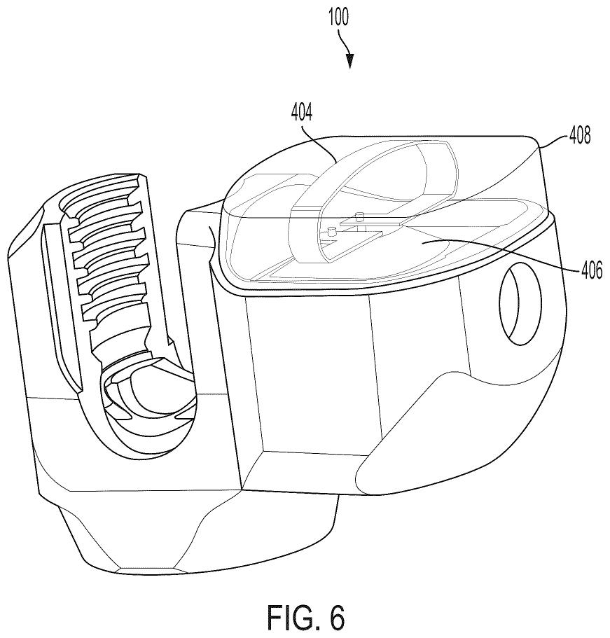

FIG. 6 shows a view of an example implant 100 showing the antenna 404 within the cap 408. As shown, the antenna 404 is on the patient-facing side of the cover 406 and receives signals from the readout electronics 450 via feedthroughs 410. In some embodiments, the cap 408 is made from a polymer or other material that avoids undue attenuation of the radio signal. In contrast, the body of the implant 100 may be formed from a metal or metal alloy, such as cobalt-chrome or titanium. The cover 406 may also be formed from a metal or metal alloy and may be epoxied or welded to the body of the implant 100, or attached in any suitable way that provides a hermetic seal of the electronics enclosure 120.

FIG. 7 illustrates an example of a surgical site (SS) monitoring system 700 that may utilize example implants 100 disclosed herein. In some embodiments, the SS monitoring system 700 may be a surgical site load monitoring system (using one or more strain gauges 302) and/or an infection monitoring system (using one or more temperature sensors). FIG. 7 illustrates a spinal-fusion construct having multiple separate sensor-equipped implants 100, each of which may have one or more sensors 302. Other embodiments within the scope of this disclosure include multiple implant systems, e.g., multiple spinal-fusion constructs, or one spinal-fusion construct and a separate sensor-equipped implant 100. Other combinations and permutations of implant systems and/or sensor-equipped implants 100 are also within the scope of the disclosure.

In one or more embodiments, the SS monitoring system 700 includes an external reader device 702 configured to receive sensor data from the implants 100. For the embodiments in which the SS monitoring system 700 includes an array of implants 100 having various MEMs sensors 302, the received data from the one or more MEMs sensors 302 may be compared to one another to diagnose the quality of the surgical procedure, the integrity of the implant 100, and/or an infection at the surgical site.

While this disclosure describes example embodiments for example fields and applications, it should be understood that the disclosure is not limited to the disclosed examples. Other embodiments and modifications thereto are possible and are within the scope and spirit of this disclosure. For example, and without limiting the generality of this paragraph, embodiments are not limited to the software, hardware, firmware, and/or entities illustrated in the figures and/or described in this document. Furthermore, embodiments (whether or not explicitly described) have significant utility to fields and applications beyond the examples described in this document.

Embodiments have been described in this document with the aid of functional building blocks illustrating the implementation of specified functions and relationships. The boundaries of these functional building blocks have been arbitrarily defined in this document for the convenience of the description. Alternate boundaries can be defined as long as the specified functions and relationships (or their equivalents) are appropriately performed. Also, alternative embodiments can perform functional blocks, steps, operations, methods, etc. using orderings different than those described in in this document.

The features from different embodiments disclosed herein may be freely combined. For example, one or more features from a method embodiment may be combined with any of the system or product embodiments. Similarly, features from a system or product embodiment may be combined with any of the method embodiments herein disclosed.

References in this document to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Furthermore, when a particular feature, structure, or characteristic is described in connection with an embodiment, it would be within the knowledge of persons skilled in the relevant art(s) to incorporate such feature, structure, or characteristic into other embodiments whether or not explicitly mentioned or described in this document. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but still co-operate or interact with each other.

The breadth and scope of this disclosure should not be limited by any of the above-described example embodiments but should be defined only in accordance with the following claims and their equivalents.

1. A load-sensing implant comprising:
an attachment portion configured to secure the implant to a bone of a subject;
a receiver portion configured to receive and secure a longitudinal member;
an electronics enclosure disposed adjacent the receiver portion and forming a common wall with the receiver portion, the electronics enclosure containing:
a sensor package comprising a plurality of strain sensors disposed on a backing, the sensor package configured to sense, via the common wall, forces applied to the receiver portion by the longitudinal member; and
support electronics configured to:
receive, from the sensor package, data representing the sensed forces, and
transmit the received data to an external device.

2. The load-sensing implant of claim 1, wherein the sensor package is disposed in a recessed pocket formed in the common wall.

3. The load-sensing implant of claim 2, wherein the recessed pocket is formed to enhance transfer of forces from the receiver portion to the sensor package.

4. The load-sensing implant of claim 1, wherein the sensor package comprises a plurality of strain gauges.

5. The load-sensing implant of claim 4, wherein the sensor package comprises two strain gauges oriented to sense shear forces and two strain gauges positioned to sense axial forces.

6. The load-sensing implant of claim 1, wherein each sensor of the sensor package has an impedance of at least 1000 ohms.

7. The load-sensing implant of claim 1, wherein the electronics enclosure is capped with a header configured to enhance wireless communication between the implant and the external device.

8. The load-sensing implant of claim 1, wherein the electronics enclosure further contains a power source.

9. The load-sensing implant of claim 1, wherein the sensor package is attached to the common wall by an adhesive applied to the backing.

10. A sensor package for a spinal implant, the sensor package comprising:
a flexible backing;
two outer strain gauges attached to the backing and having a sensing element with a longitudinal axis, the longitudinal axis oriented along a first direction;
a first inner strain gauge attached to the backing and having a sensing element with a longitudinal axis with the longitudinal axis oriented at 45 degrees from the first direction; and
a second inner strain gauge attached to the backing and having a sensing element with a longitudinal axis oriented at 45 degrees from the first direction and 90 degrees from the longitudinal axis of the sensing element of the first inner strain gauge.

11. The sensor package of claim 10, further comprising an adhesive applied to the backing.

12. The sensor package of claim 10, wherein each sensing element has an impedance of at least 1000 ohms.

13. A surgical site (SS) monitoring system comprising:
an external reader device; and
one or more load-sensing implants, each implant comprising:
an attachment portion configured to secure the implant to a bone of a subject;
a receiver portion configured to receive and secure a longitudinal member;
an electronics enclosure disposed adjacent the receiver portion and forming a common wall with the receiver portion, the electronics enclosure containing:
a sensor package comprising a plurality of strain sensors disposed on a backing, the sensor package configured to sense, via the common wall, forces applied to the receiver portion by the longitudinal member; and
support electronics configured to:
receive, from the sensor package, data representing the sensed forces, and
transmit the received data to the external reader device.

14. The system of claim 13, wherein the sensor package is disposed in a recessed pocket formed in the common wall.

15. The system of claim 14, wherein the recessed pocket is formed to enhance transfer of forces from the receiver portion to the sensor package.

16. The system of claim 13, wherein the sensor package comprises a plurality of strain gauges.

17. The system of claim 16, wherein the sensor package comprises two strain gauges oriented to sense shear forces and two strain gauges positioned to sense axial forces.

18. The system of claim 13, wherein each sensor of the sensor package has an impedance of at least 1000 ohms.

19. The system of claim 13, wherein the electronics enclosure is capped with a header configured to enhance wireless communication between the implant and the external reader device.

20. The system of claim 13, wherein at least one of the one or more load-sensing implants further comprises a temperature gauge or a position sensor.
